(19) United States
(12) Patent Application Publication
(10) Pub. No.: US 2023/0113077 A1
(43) Pub. Date: Apr. 13, 2023

(54) CONTROLLED RELEASE SELEXIPAG COMPOSITION

(71) Applicant: ACTELION PHARMACEUTICALS LTD, Allschwil (CH)

(72) Inventors: Katrien LUYTEN, Beerse (BE); Annelien VAN DROOGENBROECK, Beerse (BE)

(21) Appl. No.: 17/759,455

(22) PCT Filed: Jan. 29, 2021
(86) PCT No.: PCT/EP2021/052059

§ 371 Date: Jul. 26, 2022

(30) Foreign Application Priority Data
Jan. 31, 2020 (WO) PCT/EP2020/052519

(51) Int. Cl. A61K 31/4965; A61K 9/50; A61K 9/48; A61P 9/12

(52) U.S. Cl. CPC A61K 31/4965; A61K 9/4825; A61K 9/5015; A61K 9/5047; A61K 9/5078; A61P 9/12

(57) ABSTRACT

The present invention is concerned with controlled release compositions for oral administration comprising 2-{4-[N-(5,6-diphenylpyrazin-2-yl)-N- isopropylamino]butyloxy}-N-(methylsulfonyl)acetamide (selexipag, NS-304, ACT- 293987) and its pharmaceutically acceptable salts and/or 2-(4-((5,6-diphenylpyrazin-2-yl)(isopropyl)amino)butoxy)acetic acid (metabolite of selexipag, MRE-269, ACT- 333679) and its pharmaceutically acceptable salts; and with processes for preparing such controlled release compositions as well as to uses thereof.

### CROSS-REFERENCE TO RELATED APPLICATIONS

This application is the National Stage of International Patent Application No. PCT/EP2021/052059, filed Jan. 29, 2021, which claims the benefit of International Patent Application No. PCT/EP2020/052519, filed Jan. 31, 2020, the disclosures of which are incorporated herein by reference in their entireties.

### FIELD OF THE INVENTION

The present invention is concerned with controlled release compositions for oral administration comprising 2-{4-[N-(5,6-diphenylpyrazin-2-yl)-N-isopropylamino]butyloxy}-N-(methylsulfonyl)acetamide (selexipag, NS-304, ACT-293987), and its pharmaceutically acceptable salts, and/or 2-(4-((5,6-diphenylpyrazin-2-yl)(isopropyl)amino)butoxy)acetic acid (metabolite of selexipag, MRE-269, ACT-333679), and its pharmaceutically acceptable salts; with processes for preparing such controlled release compositions and uses thereof.

### BACKGROUND OF THE INVENTION

Selexipag and its metabolite have the following chemical structures:

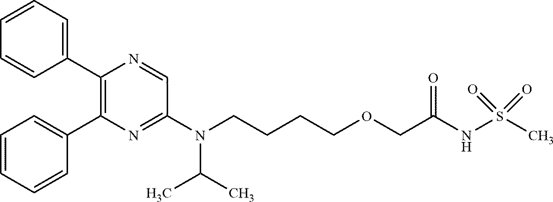 [SMILES: CC(C)N(CCCCOCC(=O)NS(C)(=O)=O)c1cnc(-c2ccccc2)c(-c2ccccc2)n1]

and

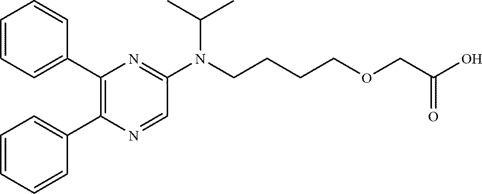 [SMILES: CC(C)N(CCCCOCC(=O)O)c1cnc(-c2ccccc2)c(-c2ccccc2)n1]

The preparation and the medicinal use of selexipag (2-{4-[N-(5,6-diphenylpyrazin-2-yl)-N-isopropylamino]butyloxy}-N-(methylsulfonyl)acetamide, NS-304, ACT-293987) and its active metabolite (2-(4-((5,6-diphenylpyrazin-2-yl)(isopropyl)amino)butoxy)acetic acid, MRE-269, ACT-333679) is described in WO2002/088084; WO2009/157396; WO2009/107736; WO2009/154246; WO2009/157397; WO2009/157398; WO2010/150865; WO2011/024874; Nakamura et al., Bioorg Med Chem (2007), 15, 7720-7725; Kuwano et al., J Pharmacol Exp Ther (2007), 322(3), 1181-1188; Kuwano et al., J Pharmacol Exp Ther (2008), 326(3), 691-699; O. Sitbon et al., N Engl J Med (2015), 373, 2522-33; Asaki et al., Bioorg Med Chem (2007), 15, 6692-6704; Asaki et al., J. Med. Chem. (2015), 58, 7128-7137. Certain formulations are disclosed in WO2013/024051, WO2014/069401, WO2018/162527 and CN107811994.

Selexipag was shown to be beneficial in the treatment of pulmonary arterial hypertension for adults. In a phase III clinical trial, among patients with pulmonary arterial hypertension, the risk of the primary composite end point of death or a complication related to pulmonary arterial hypertension was significantly lower among patients who received selexipag than among those who received placebo. Selexipag received market approval e.g. in the US and is indicated for the treatment of pulmonary arterial hypertension (PAH, WHO Group I) to delay disease progression and reduce the risk of hospitalization for PAH.

Selexipag is thought to function as a prodrug (while retaining some agonistic activity on the IP receptor on its own) which can exert long-lasting selective IP receptor agonist activity of the active metabolite 2-(4-((5,6-diphenylpyrazin-2-yl)(isopropyl)amino)butoxy) acetic acid in mammals, especially humans. The in vivo metabolism of selexipag effectively may act as a kind of ‘slow-release mechanism’ that potentially both prolongs activity and reduces typical adverse effects associated with high concentrations of PGI2 agonists (Kuwano et al., J Pharmacol Exp Ther (2007), 322(3), 1181-1188).

Adverse effects associated with PGI2 agonists are also addressed by a particular up-titration schedule. The recommended starting dose of oral selexipag for adults is 200 micrograms given twice daily. The dose is then increased in increments of 200 micrograms twice daily, usually at weekly intervals, to the highest tolerated dose up to 1600 micrograms twice daily. If a patient reaches a dose that cannot be tolerated, the dose should be reduced to the previous tolerated dose.

Selexipag is a selective IP-receptor agonist for oral use with proven efficacy and safety in adults with PAH. To date, selexipag is the only IP-receptor agonist approved globally for long-term treatment across WHO FC II-III and primarily in combination with current first-line oral PAH-specific medicines, in adult patients in need of additional therapy because of insufficient disease control. Selexipag represents an important additional treatment option for these patients.

The availability of selexipag, a highly selective IP-receptor agonist for oral use and with demonstrated benefit on PAH disease outcomes in add-on therapy, provides an important rationale to initiate a prostacyclin-pathway therapy at a medically appropriate stage of PAH disease, without major consequences for the patient’s lifestyle.

So far, standard film-coated tablet formulations of selexipag intended for twice daily oral administration have been used, wherein excipients comprise D-mannitol, corn starch, low substituted hydroxypropylcellulose, hydroxypropylcellulose, and magnesium stearate; and the tablets are film coated with a coating material containing hypromellose, propylene glycol, titanium dioxide, carnauba wax along with mixtures of iron oxides.

However, it is of advantage for the patients if the administration of selexipag or its metabolite could be reduced to one administration per day. Therefore, there is a need to develop formulations of selexipag or its metabolite which provide a slow or controlled release of the active pharmaceutical ingredient.

### SUMMARY OF THE INVENTION

It is the object of the present invention to provide a controlled release formulation of selexipag and/or its metabolite.

The present inventors have found that a specific coating provides such controlled release of the active pharmaceutical ingredient. Moreover, it has been found that further protective coatings may provide advantages in respect of stability of the active pharmaceutical ingredient.

The present invention therefore relates to a pharmaceutical formulation comprising a drug core and a release rate controlling membrane, optionally comprising one or more protecting coats. Moreover, the present invention relates to pharmaceutical dosage forms comprising said formulation for sustained drug release in a patient.

### DESCRIPTION OF THE FIGURES

FIG. 1 shows a graph of an in vitro dissolution test of 400 microgram selexipag capsules containing the pharmaceutical formulation with 3 different amounts of release rate controlling membranes. Dissolution testing was performed in 0.05 M sodium phosphate buffer pH6.8 using the paddle apparatus at 37° C.

FIG. 2 shows a graph of an in vitro dissolution test of a 400 microgram selexipag metabolite capsule containing the pharmaceutical formulation with a release rate controlling membrane. Dissolution testing was performed in 0.05 M sodium phosphate buffer pH6.8 using the paddle apparatus at 37° C.

### DETAILED DESCRIPTION OF THE INVENTION

The present invention relates to a pharmaceutical formulation comprising
particles comprising as active pharmaceutical ingredient 2-{4-[N-(5,6-diphenylpyrazin-2-yl)-N-isopropylamino]butyloxy}-N-(methylsulfonyl)acetamide (selexipag), or pharmaceutically acceptable salts thereof, and/or 2-(4-((5,6-diphenylpyrazin-2-yl)(isopropyl)amino)butoxy)acetic acid (selexipag metabolite), or pharmaceutically acceptable salts thereof, and a water soluble polymer, to form a drug core;
said particles being coated with a release rate controlling membrane coating comprising ethyl cellulose (EC) and hydroxypropyl methylcellulose (HPMC); and
optionally one or more protective coats.

The pharmaceutical formulation of the present invention is composed of particles which comprise the pharmaceutically active ingredient and a release rate controlling membrane coating. Additional coatings, such as, but not limited to, protective coatings, may optionally be applied to the particles.

In the present context and if not explicitly described otherwise, the term “active pharmaceutical ingredient (API)” encompasses 2-{4-[N-(5,6-diphenylpyrazin-2-yl)-N-isopropylamino]butyloxy}-N-(methylsulfonyl)acetamide (selexipag) in free form and/or a pharmaceutically acceptable salt thereof, and/or 2-(4-((5,6-diphenylpyrazin-2-yl)(isopropyl)amino)butoxy)acetic acid (selexipag metabolite) in free form and/or a pharmaceutically acceptable salt thereof.

In one embodiment, the pharmaceutical formulation comprises 2-{4-[N-(5,6-diphenylpyrazin-2-yl)-N-isopropylamino]butyloxy}-N-(methylsulfonyl)acetamide (selexipag) in free form as active pharmaceutical ingredient.

In one embodiment, the pharmaceutical formulation comprises 2-(4-((5,6-diphenylpyrazin-2-yl)(isopropyl)amino)butoxy)acetic acid (selexipag metabolite) in free form, and/or a pharmaceutically acceptable salt thereof as active pharmaceutical ingredient.

In one embodiment, the pharmaceutical formulation comprises 2-(4-((5,6-diphenylpyrazin-2-yl)(isopropyl)amino)butoxy)acetic acid (selexipag metabolite) in free form as active pharmaceutical ingredient.

In one embodiment, the pharmaceutical formulation comprises 2-{4-[N-(5,6-diphenylpyrazin-2-yl)-N-isopropylamino]butyloxy}-N-(methylsulfonyl)acetamide (selexipag) and 2-(4-((5,6-diphenylpyrazin-2-yl)(isopropyl)amino)butoxy)acetic acid (selexipag metabolite), and/or pharmaceutically acceptable salts thereof.

The term “pharmaceutically acceptable salts” refers to salts that retain the desired biological activity of selexipag or its metabolite, and exhibit minimal undesired toxicological effects. Such salts include inorganic or organic acid and/or base addition salts. For reference see for example “Handbook of Pharmaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quéré (Eds.), RSC Publishing, 2012.

In this invention, Selexipag is preferably present in its amorphous state.

The particles of the pharmaceutical formulation comprise a drug core, i.e. the core of the particle comprises the active pharmaceutical ingredient, either in free form or as a pharmaceutically acceptable salt thereof. Thereby, the drug core further comprises at least one pharmaceutically acceptable excipient, such as a water-soluble polymer. The invention encompasses particles wherein the core itself is formed by the active pharmaceutical ingredient and a pharmaceutically acceptable excipient, such as a water-soluble polymer. Preferably, however, the invention encompasses particles wherein the drug core is an inert particle coated by a drug coat comprising the active pharmaceutical ingredient and a water-soluble polymer. Thereby, the water-soluble polymer is a polymer as further described below. In this context, the term “drug core” and “drug coated core” are used as synonyms.

It has been found that the specific mixture of ethyl cellulose (EC) and hydroxypropyl methylcellulose (HPMC) is particularly useful for the formation of the release rate controlling membrane coating.

Ethyl cellulose (EC) is an ethyl cellulose polymer derived from cellulose and bearing the backbone of cellulose. Cellulose is first treated with an alkaline solution to produce alkali cellulose, which is subsequently reacted with ethyl chloride, yielding in crude ethyl cellulose, which is a water-insoluble polymer. Preferred ethyl cellulose (EC) has an ethoxyl content of 48.0 to 49.5%. Ethyl cellulose can be produced in a number of different viscosities. Viscosity increases as the length of the molecule increases. Suitable EC include those having a viscosity from 3 to 110 mPa.s, preferably viscosity of 16 to 24 mPa.s, for example 20 mPa.s, preferably indicated as nominal viscosity.

Particularly preferred is ethyl cellulose (EC) with a viscosity in the range of 16 to 24 mPa.s (cP), i.e. a nominal Ubbelohde viscosity of 16 to 24 mPa.s for a 5% solution (in 80% toluene and 20% ethanol) measured with 25° C. The ethoxyl content is as described above, preferably 48.0 to 49.5 %. An example for such ethyl cellulose (EC) is designated herein as ethyl cellulose (EC) 20 (the term 20 referring to the nominal viscosity in mPa.s). An example for ethyl cellulose (EC) 20 is Ethocel™ standard premium 20.

Hydroxypropyl methylcellulose (HPMC) or hypromellose (INN, see Martindale, The Extra Pharmacopoeia, 29th edition, page 1435) is a methyl cellulose substituted with propylene oxide. Various degrees of polymerization and substitution are known. Suitable HPMC contains sufficient hydroxypropyl and methoxy groups to render it water-soluble. The methoxy degree of substitution refers to the average number of methyl ether groups present per anhydroglucose unit of the cellulose molecule. The hydroxypropyl molar substitution refers to the average number of moles of propylene oxide which have reacted with each anhydroglucose unit of the cellulose molecule.

Preferably hydroxypropyl methylcellulose with low viscosity, i.e. 5 mPa.s, is used, e.g. hydroxypropyl methylcellulose 2910 5 mPa.s. In the four digit number “2910”, the first two digits represent the approximate percentage of methoxyl groups and the third and fourth digits the approximate percentage composition of hydroxypropoxyl groups. 5 mPa.s is a value indicative of the apparent viscosity of a 2% aqueous solution at 20° C. More precisely, such hydroxypropyl methylcellulose 2910 5 mPa.s has a methoxyl substitution of 28 to 30% (represented as “29”), and a hydroxypropyl substitution of 7.0 to 12.0 % (represented as “10”).

Suitable HPMC include those having a viscosity from 1 to 100 mPa.s, in particular from 3 to 15 mPa.s, preferably 5 mPa.s The most preferred type of HPMC having a viscosity of 5 mPa.s., is the commercially available HPMC 2910 5 mPa.s.

The weight-by-weight ratio of ethyl cellulose (EC) to hydroxypropyl methylcellulose (HPMC) in the release rate controlling membrane coating may range from 95 : 5 to 50 : 50. Suitable upper limits of the weight-by-weight ratios of ethyl cellulose (EC) to hydroxypropyl methylcellulose (HPMC) are 95 : 5, 90: 10, 85 : 15. Suitable lower limits of the weight-by-weight ratios of ethyl cellulose (EC) to hydroxypropyl methylcellulose (HPMC) are 50 : 50, 55 : 45, 60 : 40, and 65 : 35. It is to be understood that each upper limit may be combined with each lower limit in order to define the range of ethyl cellulose (EC) to hydroxypropyl methylcellulose (HPMC). Specifically, the ranges may be 95 : 5 to 50 : 50 of EC to HPMC (w/w); 95 : 5 to 55 : 45 of EC to HPMC (w/w); 95 : 5 to 60 : 40 of EC to HPMC (w/w); 95 : 5 to 65 : 35 of EC to HPMC (w/w); 90: 10 to 50 : 50 of EC to HPMC (w/w); 90: 10 to 55 : 45 of EC to HPMC (w/w); 90: 10 to 60 : 40 of EC to HPMC (w/w); 90: 10 to 65 : 35 of EC to HPMC (w/w); 85 : 15 to 50 : 50 of EC to HPMC (w/w); 85 : 15 to 55 : 45 of EC to HPMC (w/w); 85 : 15 to 60 : 40 of EC to HPMC (w/w); 85 : 15 to 65 : 35 of EC to HPMC (w/w); 80 : 20 to 50 : 50 of EC to HPMC (w/w); 80 : 20 to 55 : 45 of EC to HPMC (w/w); 80 : 20 to 60 : 40 of EC to HPMC (w/w); or 80 : 20 to 65 : 35 of EC to HPMC (w/w). Preferred ranges in particular for selexipag are for example 95 : 5 to 50 : 50 of EC to HPMC (w/w); 90: 10 to 55 : 45 of EC to HPMC (w/w); 85 : 15 to 60 : 40 of EC to HPMC (w/w); 80 : 20 to 60 : 40 of EC to HPMC (w/w); or 80 : 20 to 65 : 35 of EC to HPMC (w/w). A preferred example for selexipag is a weight-by-weight ratio of ethyl cellulose (EC) : hydroxypropyl methylcellulose (HPMC) of 75 : 25 and 90:10, particularly preferred is 75 : 25.

The release rate controlling membrane coating may comprise a plasticizer. A plasticizer increases flexibility and integrity in ethyl cellulose films. Cracks formation during coating, drying, or upon storage could expose the drug from the layer underneath in the beads, resulting e.g. in a change in release profile, a burst, chemical stability issues. Besides the improvement in the film mechanical properties, a plasticizer can influence the drug release through the film.

The plasticizer is selected from the group comprising dibutyl sebacate, diethyl phthalate,triethyl citrate and triacetin. Dibutyl sebacate is a preferred plasticizer.

The weight percentage of plasticizer in the release rate controlling membrane coating is 10 to 40 wt%. As an example for calculation, if the content of EC and HPMC together would be 100 mg, then 20 wt% of plasticizer would be 20 mg, all together this would sum up to 120 mg. In one embodiment, the plasticizer dibutyl sebacate is contained in the release rate controlling membrane coating from 10 wt% to 30 wt%, preferably from 15 to 25 wt%, for example 18 to 22 wt%, most preferably 20 wt% in relation to the EC/HPMC content of the release rate controlling membrane coating.

The weight of the ethyl cellulose (EC) plus hydroxypropyl methylcellulose (HPMC) in the release rate controlling membrane coating ranges from 5 wt% to 50 wt% based on the weight of the drug core.

Preferably, the weight of the ethyl cellulose (EC) plus hydroxypropyl methylcellulose (HPMC) in the release rate controlling membrane coating ranges from 5 wt% to 40 wt%, or from 5 wt% to 35 wt%, for example from 8 wt% to 35 wt%, or from 10 wt% to 30 wt%, such as for instance 10 wt%, 20 wt% or 30 wt% based on the weight of the drug core. This is also referred to as “weight gain” in the examples below. It is calculated with the theoretical weight of the drug core. The rate of release of the active ingredient from the particles is approximately inversely proportional with the thickness of the release rate controlling membrane coating. In addition to the ethyl cellulose (EC) and hydroxypropyl methylcellulose (HPMC), the release rate controlling membrane may further comprise additional excipients, for example a plasticizer, as described above. It is to be understood that in such a case, the weight gain of the complete release rate controlling membrane coating is proportionally higher.

The release rate controlling membrane coating may be applied to the drug core as a solution or dispersion in an organic solvent system. A useful organic system comprises a chlorinated hydrocarbon, acetone and/or an alcohol, or mixtures thereof. The chlorinated hydrocarbon is preferably dichloromethane. The alcohol may be selected from the group consisting of methanol, ethanol or isopropanol. Preferably, the alcohol is methanol or ethanol. Preferably, the release rate controlling membrane coating is applied to the drug cores in an organic solvent system comprising a chlorinated hydrocarbon and an alcohol, for instance dichloromethane and ethanol or dichloromethane and methanol. Particularly preferred is dichloromethane/ethanol in a ratio of 50/50 (m/m).

Optionally, a protective coat lies between the drug core and the release rate controlling membrane coating. This protective coat may be a light protective coat or a seal coat. For instance, a seal coating polymer layer is applied to the drug coated cores to prevent sticking of the particles during the process and to prevent migration of the drug into the release rate controlling membrane. Preferably, a thin layer of HPMC 2910 5 mPa.s and polyethylene glycol (PEG), in particular polyethylene glycol 400 is used as a seal coating polymer layer. Moreover, pigments may be applied to the protective coat for light protection. Such pigments may be iron oxide(s), for instance iron oxide red and/or titanium dioxide.

In the following, the drug core, i.e. the particles comprising as active pharmaceutical ingredient 2-{4-[N-(5,6-diphenylpyrazin-2-yl)-N-isopropylamino]butyloxy}-N-(methylsulfonyl)acetamide (selexipag), or pharmaceutically acceptable salts thereof, and/or 2-(4-((5,6-diphenylpyrazin-2-yl)(isopropyl)amino)butoxy)acetic acid (selexipag metabolite), or pharmaceutically acceptable salts thereof, and a water soluble polymer; is described.

The water-soluble polymer can conveniently be a film forming polymer. Useful water-soluble polymers are polymers that have an apparent viscosity of 1 to 100 mPa.s. Viscosity is thereby measured in medium appropriate for the specific polymer. For instance, the viscosity of HPMC may be measured in a 2% aqueous solution at 20° C.

Preferably, the water-soluble polymer is selected from the group consisting of
hydroxyalkyl alkylcelluloses, preferably hydroxyethyl methylcellulose and hydroxypropyl methylcellulose (HPMC);
hydroxypropyl methylcellulose acetate succinate (HPMC-AS);
hydroxypropyl methyl cellulose phthalate (HPMCP)
alkylcelluloses, preferably methylcellulose;
hydroxyalkylcelluloses, preferably hydroxymethylcellulose, hydroxyethylcellulose, hydroxypropylcellulose (HPC) and hydroxybutylcellulose;
carboxyalkylcelluloses, preferably carboxymethylcellulose;
alkali metal salts of carboxyalkylcelluloses, preferably sodium carboxymethylcellulose;
carboxyalkylalkylcelluloses, preferably carboxymethylethylcellulose;
carboxyalkylcellulose esters;
starches;
pectines, preferably carboxymethylamylopectine;
chitine derivates, preferably chitosan;
polysaccharides, preferably alginic acid, alkali metal and ammonium salts thereof, carrageenans, galactomannan, traganth, agar agar, gummi arabicum, guar gummi and xanthan gummi;
polyacrylic acids and salts thereof;
polymethacrylic acids and salts thereof, methacrylate copolymers;
polyvinylalcohol (PVA);
polyvinylpyrrolidone, copolymers of polyvinylpyrrolidone with vinyl acetate; and
polyalkylene oxides, preferably polyethylene oxide and polypropylene oxide and copolymers of ethylene oxide and propylene oxide.

Non-enumerated polymers which are pharmaceutically acceptable and have appropriate physico-chemical properties as defined hereinbefore are equally suited for preparing particles according to the present invention.

A suitable hydroxypropylcellulose (HPC) includes those having a viscosity from 30 to 900 mPa.s in a 5-10% aqueous solution.

A suitable hydroxypropyl methyl cellulose phthalate (HPMCP) includes HPMCP HP-50 and HP-55. Various grades of hypromellose phthalate are available with differing degrees of substitution and physical properties, grades HP-50 and HP-55 are preferred. The number following ‘HP’ in each grade designation refers to the pH value (x10) at which the polymer dissolves in aqueous buffer solutions.

A suitable polyvinylalcohol (PVA) includes those having a viscosity from 4 to 7 mPa.s for a 4% w/v aqueous solution at 20° C., preferably 5 mPa s. The most preferred type of PVA has a percentage of hydrolysis greater than about 86.5 mol%, preferably 86.5 to 89.0 mol%.

Preferably, the water-soluble polymer is selected from the group consisting of
hydroxypropyl methylcellulose (HPMC); and
hydroxypropyl methylcellulose acetate succinate (HPMC-AS).

Suitable hydroxypropyl methylcellulose (HPMC) include those having a viscosity from 1 to 100 mPa.s, in particular from 3 to 15 mPa.s, preferably 5 mPa.s when dissolved in a 2% aqueous solution at 20° C. solution. The most preferred type of HPMC having a viscosity of 5 mPa.s., is the commercially available HPMC 2910 5 mPa.s. As mentioned before, such HPMC 2910 5 mPa.s has a methoxyl substitution of 28 to 30% (represented as “29”), and a hydroxypropyl substitution of 7.0 to 12.0% (represented as “10”).

A suitable hydroxypropyl methylcellulose acetate succinate (HPMC-AS) includes those having a viscosity from 2.4 to 3.6 mPa s in a 2% aqueous solution at 20° C. The most preferred type of HPMC-AS having a nominal viscosity of 3 mPa.s., is the commercially available AQOAT. Hypromellose acetate succinate is available in several grades, according to the content of acetyl and succinoyl groups, pH at which the polymer dissolves and its predominant particle size. For example, a suitable HPMC-AS may contain NLT 12.0% and NMT 28.0% of methoxy groups (-OCH3), NLT 4.0% and NMT 23.0% of hydroxypropoxy groups (-OCH2CHOHCH3), NLT 2.0% and NMT 16.0% of acetyl groups (-COCH3), and NLT 4.0% and NMT 28.0% of succinoyl groups (-COC2H4COOH), calculated on the dried basis.

Thereby, hydroxypropyl methylcellulose (HPMC) with the same properties as described above is particularly preferred, i.e. hydroxypropyl methylcellulose 2910 5 mPa.s with a methoxyl substitution of 28 to 30% (represented as “29”), and a hydroxypropyl substitution of 7.0 to 12.0% (represented as “10”).

The weight-by-weight ratio of the active pharmaceutical ingredient in free form to the water-soluble polymer as described above is in the range of 1 : 0.5 to 1 : 100. Preferably, the weight-by-weight ratio of the active pharmaceutical ingredient in free form to the water-soluble polymer is in the range of 1 : 1 to 1 : 50, in the range of 1 : 1 to 1 : 40, in the range of 1 : 1 to 1 : 35, for example in the range of 1 : 1 to 1 : 30.

The term “active pharmaceutical ingredient in free form” means 2-{4-[N-(5,6-diphenylpyrazin-2-yl)-N-isopropylamino]butyloxy}-N-(methylsulfonyl)acetamide (selexipag), or 2-(4-((5,6-diphenylpyrazin-2-yl)(isopropyl)amino)butoxy)acetic acid (selexipag metabolite) in free form, i.e. not as a salt. In case that a pharmaceutically acceptable salt of these compounds is used, the weight of such salt is proportionally higher, depending on the molecular weight of the salt. In other words, the weight-by-weight ratio is always calculated with the free form as a basis, and in case a pharmaceutically acceptable salt is used, its weight is proportionally higher.

Particularly preferably, the weight-by-weight ratio of the active pharmaceutical ingredient in free form to HPMC as water-soluble polymer as described above is in the range of 1 : 0.5 to 1 : 100. Preferably, the weight-by-weight ratio of the active pharmaceutical ingredient in free form to HPMC as water-soluble polymer is in the range of 1 : 1 to 1 : 50, in the range of 1 : 1 to 1 : 40, in the range of 1 : 1 to 1 : 35, for example in the range of 1 : 1 to 1 : 30.

The weight-by-weight ratio of active pharmaceutical ingredient to other water-soluble polymers may be determined by a person skilled in the art by straightforward experimentation. The lower limit is determined by practical considerations.

As described above, in one embodiment, the active pharmaceutical ingredient and the water-soluble polymer is layered or coated on an inert sphere. Thereby, the active pharmaceutical ingredient and the water-soluble polymer are as described above, including their preferred embodiments.

The inert spheres of the formulation are spheres having a diameter of 250-1180 micrometer. Preferably, the diameter is 400 to 900 micrometer, for example 425 to 850 micrometer, or 500 to 710 micrometer. For instance, a nominal particle size of 600 micrometer refers to a particle size distribution specification of ≥75% 500-710 micrometer beads, ≤10% >710 micrometer beads and ≤15% <500 micrometer beads.

Pellets, beads or cores of the dimensions mentioned herein can be obtained by sieving through nominal standard test sieves as described in the CRC Handbook, 64th ed., page F-114. Nominal standard sieves are characterized by the mesh/hole width (micrometer), DIN 4188 (mm), ASTM E 11-70 (No), Tyler™ (mesh) or BS 410 (mesh) standard values. Throughout this description and the claims, particle sizes are designated by reference to the mesh/hole width in micrometer and to the corresponding Sieve No in the ASTM E11-70 standard.

Materials suitable for use as cores in the particles according to the present invention are manifold, provided that said materials are pharmaceutically acceptable and have appropriate dimensions as described above and firmness. Examples of such materials are polymers e.g. plastic resins; inorganic substances, e.g. silica, glass, hydroxyapatite, salts (sodium or potassium chloride, calcium or magnesium carbonate) and the like; organic substances, e.g. activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides.

Preferred are saccharides, more preferred are microcrystalline cellulose, sugar and isomalt spheres, in particular microcrystalline cellulose as microcrystalline cellulose spheres.

Microcrystalline cellulose (MCC) spheres are particularly preferred, for its low reactivity, insolubility in the solvents used for drug layering, high sphericity, fine and uniform particle size, high mechanical strength, good processability.

As an alternative to the drug layered or drug coated inert pellets or spheres described herein, suitable particles comprising the active pharmaceutical ingredient may also be formed by granules or by spheroids (spherical granules) prepared according to art-known methods of granulation and spheronization.

In case that the active pharmaceutical ingredient and the water-soluble polymer is layered or coated on an inert sphere, a drug coating solution is prepared by dissolving into a suitable solvent system appropriate amounts of active pharmaceutical ingredient and a water-soluble polymer. A suitable solvent system comprises an organic solvent such as a chlorinated hydrocarbon or acetone, and/or an alcohol. Preferred drug coating solvents are dichloromethane, acetone, methanol, ethanol and isopropanol. More preferred is a mixture of a chlorinated hydrocarbon and an alcohol. Particularly preferred are instance dichloromethane and methanol or dichloromethane and ethanol.

The ratio between chlorinated hydrocarbon and alcohol may range from 5 : 1 to 1 : 5, or from 3 : 1 to 1 : 3, or from 2 : 1 to 1 : 2, or from 1.5 : 1 to 1 : 1.5, all in m/m. Particularly preferred is a ratio of 1 : 1 m/m. Ethanol may be denatured, for example, with butanone.

The amounts of solids, i.e. active pharmaceutical ingredient and water-soluble polymer, in the drug coating solution may range from 1% to 15% (w/w) and preferably 2% to 8%. The solution is preferably stirred during the coating process.

In addition, the particles according to the present invention may further contain various additives such as thickening agents, lubricants, surfactants, preservatives, complexing and chelating agents, electrolytes or other active ingredients.

Optionally, a protective coat lies between the drug core and the release rate controlling membrane coating Moreover, the protective coat can be placed on the release rate controlling membrane coating.

This protective coat may be a light protective coat and/or a seal coat, but it may also be a further functional coat such as an enteric coat. Enteric coats are known in the art.

The pharmaceutical formulation according to the present invention, i.e. the particles comprising the active pharmaceutical ingredient and a water-soluble polymer, said particles being coated with a release rate controlling membrane coating as described above, can be part of a pharmaceutical dosage form. Such pharmaceutical dosage form comprises a therapeutically effective amount of the pharmaceutical formulation as described herein. The pharmaceutical dosage form may be a capsule, into which the pharmaceutical formulation is filled in. Thereby, the capsule may additionally serve to protect the pharmaceutical formulation therein, for instance by light protection. This can be achieved by dying the capsule, for instance with pigments, such as iron oxide(s), for instance iron oxide red, or titanium dioxide, but also with light protective organic compounds.

The therapeutically effective amount may be 20 to 3500 microgram/day, i.e. the dosage form, which may be a capsule, may contain 20 to 3500 microgram, preferably 400 microgram to 3200 microgram. Preferable amounts per dosage form, which may be a capsule, are 400 microgram, 600 microgram, 800 microgram, 1000 microgram, 1200 microgram, 1400 microgram, 1600 microgram, 1800 microgram, 2000 microgram, 2200 microgram, 2400 microgram, 2600 microgram, 2800 microgram, 3000 microgram, and 3200 microgram.

The pharmaceutical formulation and/or the dosage form comprising it, is particularly suitable for oral administration once daily. In other words, the present invention relates to a pharmaceutical formulation and/or a dosage form comprising it, for use in the prevention or treatment of the diseases as described herein, wherein a therapeutically effective amount of said formulation or dosage form is administered orally, preferably once daily.

The pharmaceutical formulation and/or the dosage form comprising it according to the present invention preferably delivers a therapeutically effective amount of active pharmaceutical ingredient to a patient during the 24 hours following a single once daily administration.

Further, the pharmaceutical formulation and/or the dosage form of the present invention is suitable for colon delivery.

Preferably, the pharmaceutical dosage form is a capsule, preferably a hard-gelatin capsule. Further preferred are capsules, e.g. hard-gelatin capsules comprising light protecting substances, for instance pigments, such as for example iron oxides, for instance iron oxide red, and titanium dioxide. Thereby, the particles of the pharmaceutical formulation may be filled in capsules, preferably hard-gelatin capsules, using standard automatic capsule filling machines. Suitable earthing and de-ionisation equipment can advantageously prevent development of electrostatic charges.

The present invention also concerns pharmaceutical packages suitable for commercial sale comprising a container, a formulation of the pharmaceutical active ingredient/dosage form as described hereinabove and associated with said package written matter specifying how said formulation should be administered.

Said pharmaceutical packages may comprise the target dose of the active pharmaceutical ingredient, i.e. packaged into daily doses. They may also be adapted for titrating a patient who is ‘IP receptor agonist’-naïve, i.e. a patient who has not been exposed to an IP receptor agonist before and who should start with small, well-tolerated doses before being exposed to ever higher doses until the optimal dose is reached.

Said dose up-titration may thereby include a starting dose strength of 400 microgram/day and is up-titrated up to 3200 microgram/day in 400 microgram increments. In other words, the pharmaceutical package may by adapted for titration a patient from 400 microgram/day, to 800 microgram/day, to 1200 microgram/day, to 1600 microgram/day, to 2000 microgram/day, to 2400 microgram/day, to 2800 microgram/day, up to a dose of 3200 microgram/day. Patients are up-titrated to the personal maximum tolerated maintenance dose on which they will stay. This instruction may be included in the leaflet.

Further, the present invention relates to a process for preparing a pharmaceutical formulation as described herein, comprising
(a) admixing the active pharmaceutical ingredient 2-{4-[N-(5,6-diphenylpyrazin-2-yl)-N-isopropylamino]butyloxy}-N-(methylsulfonyl)acetamide (selexipag), or pharmaceutically acceptable salts thereof, and/or 2-(4-((5,6-diphenylpyrazin-2-yl)(isopropyl)amino)butoxy)acetic acid (selexipag metabolite), or pharmaceutically acceptable salts thereof, with a water-soluble polymer in an organic solvent to form a drug core;
(b) optionally applying a protective coat to the drug core;
(c) admixing ethyl cellulose (EC) and hydroxypropyl methylcellulose (HPMC), and optionally a plasticizer, in an organic solvent, and applying the release rate controlling membrane coating;
(d) optionally applying a protective coat to the particle comprising the release rate controlling membrane coating.

Preferably, in step (a), the active pharmaceutical ingredient 2-{4-[N-(5,6-diphenylpyrazin-2-yl)-N-isopropylamino]butyloxy}-N-(methylsulfonyl)acetamide (selexipag), or pharmaceutically acceptable salts thereof, and/or 2-(4-((5,6-diphenylpyrazin-2-yl)(isopropyl)amino)butoxy)acetic acid (selexipag metabolite), or pharmaceutically acceptable salts thereof, is admixed with a water-soluble polymer in an organic solvent, and applied to a neutral or inert particle. This means that a drug core is formed by applying a mixture of the active pharmaceutical ingredient and the water-soluble polymer to an inert particle.

Moreover, the present invention relates to a pharmaceutical formulation obtainable by the above process. The process involves the use of organic solvents. Preferred organic solvents or solvent systems are those described herein in respect of the preparation of the drug core / drug coat, as well as those described for the release rate controlling membrane coating. As the release rate controlling membrane coating comprises ethyl cellulose (EC), the film structure can differ depending on the solvent system used, i.e. aqueous versus organic solvent system.

Hence, the particles according to the present invention are conveniently prepared in the following manner. A drug coating solution is prepared by dissolving into a suitable organic solvent system appropriate amounts of active pharmaceutical ingredient and a water-soluble polymer. A suitable solvent system comprises an organic solvent such as a chlorinated hydrocarbon and/or an alcohol as described above, for example a mixture of dichloromethane and methanol. The amounts of solids, i.e. active pharmaceutical ingredient and water-soluble polymer, in the drug coating solution may range from 1% to 15% (w/w) and preferably 2 % to 8 % (w/w). The solution is preferably stirred during the coating process.

The drug coating process (on an industrial scale) is conveniently conducted in a fluidized bed granulator (e.g. Glatt type WSG-30 or GPCG-30) equipped with a Wurster bottom spray insert (e.g. an 18 inch Wurster insert). Laboratory scale process development can be performed on a GCPG-2 with a 4 inch Wurster bottom insert and a Mini Glatt. Obviously the process parameters depend on the equipment used.

The spraying rate should be regulated carefully. Too low a spraying rate can cause some spray drying of the drug coating solution and result in a loss of product. Too high a spraying rate will cause overwetting with subsequent agglomeration. Agglomeration being the most serious problem, lower spraying rates may be used initially, to be increased as the coating process proceeds and the particles grow larger.

The atomizing air pressure with which the drug coating solution is applied also influences the coating performance. Low atomizing air pressure results in the formation of larger droplets and an increased tendency toward agglomeration. High atomizing air pressure could conceivably carry the risk of spray drying the drug solution, but this was found not to be a problem. Consequently, atomizing air pressure may be set at nearly maximum levels.

Fluidizing air volume should be set in such a manner that optimum pellet circulation is obtained. Too low an air volume will cause insufficient fluidization of the pellets; too high an air volume will interfere with the pellet circulation due to countercurrent air streams developing in the apparatus.

The coating process is advantageously conducted by employing an inlet-air temperature ranging from about 40° C. to about 65° C. Higher temperatures may speed up the process but have the disadvantage that solvent evaporation is so rapid that the coating liquid is not spread uniformly on the surface of the pellets resulting in the formation of a drug coating layer with high porosity. As the bulk volume of the coated pellets increases, drug dissolution may decrease significantly to unacceptable levels. Obviously, the optimum process temperature will further depend on the equipment used, the nature of the core, the batch volume, the solvent and the spraying rate.

The release rate controlling membrane coating polymer layer is applied to the drug (or seal) coated cores in a fluidized bed granulator with Wurster bottom spray insert. The release rate controlling membrane coating suspension or solution can be prepared by suspending or dissolving an appropriate amount of a release rate controlling membrane coating polymer into a suitable solvent system. Such a system, is, e.g. a chlorinated hydrocarbon, acetone and/or an alcohol, preferably a mixture of a chlorinated hydrocarbon and an alcohol, such as for instance dichloromethane and methanol or ethanol, preferably dichloromethane and ethanol. The ratio between chlorinated hydrocarbon and alcohol may range from 5 : 1 to 1 : 5 or any of the ratios as described above, preferred is a ratio of 1.5 : 1 (m/m). Ethanol may be denatured, for example, with butanone.

The amount of release rate controlling membrane coating polymer in the spraying suspension or solution may range from 1 to 10% (w/w), preferably 3 to 8% (w/w), and more preferably 3.5 to 7.5% (w/w). The release rate controlling membrane coating spraying suspension or solution is advantageously stirred during the spraying process. The parameter setting for conducting this last step is essentially similar to that used in the previous coating processes.

All coating processes are preferably conducted under an inert atmosphere of e.g. nitrogen. The coating equipment should preferably be grounded and provided with an appropriate solvent recovery system containing an efficient condensing system.

In order to decrease residual solvent levels in the pellets following the application of the release rate controlling membrane coating from an organic solution, the pellets can conveniently be dried in any suitable drying apparatus. After drying, the particles may be sieved.

The pharmaceutical formulation or dosage form as described hereinabove is suitable for use in the prevention and/or treatment of ulcer, digital ulcer, diabetic gangrene, diabetic foot ulcer, pressure ulcer (bedsore), hypertension, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance (e.g., chronic arterial occlusion, intermittent claudication, peripheral embolism, vibration syndrome, Raynaud’s disease), connective tissue disease (e.g., systemic lupus erythematosus, scleroderma, mixed connective tissue disease, vasculitic syndrome), reocclusion/restenosis after percutaneous transluminal coronary angioplasty (PTCA), arteriosclerosis, thrombosis (e.g., acute-phase cerebral thrombosis, pulmonary embolism), transient ischemic attack (TIA), diabetic neuropathy, ischemic disorder (e.g., cerebral infarction, myocardial infarction), angina (e.g., stable angina, unstable angina), chronic kidney diseases including glomerulonephritis and diabetic nephropathy at any stage, allergy, bronchial asthma, restenosis after coronary intervention such as atherectomy and stent implantation, thrombocytopenia by dialysis, the diseases in which fibrosis of organs or tissues is involved [e.g., renal diseases such as tubulointerstitial nephritis), respiratory diseases (e.g., interstitial pneumonia, (idiopathic) pulmonary fibrosis, chronic obstructive pulmonary disease), digestive diseases (e.g, hepatocirrhosis, viral hepatitis, chronic pancreatitis and scirrhous stomachic cancer), cardiovascular diseases (e.g, myocardial fibrosis), bone and articular diseases (e.g, bone marrow fibrosis and rheumatoid arthritis), skin diseases (e.g, cicatrix after operation, scalded cicatrix, keloid, and hypertrophic cicatrix), obstetric diseases (e.g., hysteromyoma), urinary diseases (e.g., prostatic hypertrophy), other diseases (e.g., alzheimer’s disease, sclerosing peritonitis, type I diabetes and organ adhesion after operation)], erectile dysfunction (e.g., diabetic erectile dysfunction, psychogenic erectile dysfunction, psychotic erectile dysfunction, erectile dysfunction associated with chronic renal failure, erectile dysfunction after intrapelvic operation for removing prostata, and vascular erectile dysfunction associated with aging and arteriosclerosis), inflammatory bowel disease (e.g., ulcerative colitis, Crohn’s disease, intestinal tuberculosis, ischemic colitis and intestinal ulcer associated with Behcet disease), gastritis, gastric ulcer, ischemic ophthalmopathy (e.g., retinal artery occlusion, retinal vein occlusion, ischemic optic neuropathy), sudden hearing loss, avascular necrosis of bone, intestinal damage caused by administration of a non-steroidal anti-inflammatory agent and symptoms associated with lumbar spinal canal stenosis.

Preferably, said pharmaceutical formulation or dosage form may be used in the prevention or treatment of ulcer, digital ulcer, diabetic gangrene, diabetic foot ulcer, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance, connective tissue disease, chronic kidney diseases including glomerulonephritis and diabetic nephropathy at any stage, diseases in which fibrosis of organs or tissues is involved, or respiratory diseases.

Preferably, said pharmaceutical formulation or dosage form may be used in the prevention or treatment of pulmonary arterial hypertension (PAH).

It is to be understood that the pharmaceutical composition according to any one of the preceding embodiments may be used for the manufacture of a medicament, in particular for a medicament for preventing and/or treating the above-referenced indications.

It is further to be understood that the present invention also relates to a method for preventing and/or treating the above-referenced diseases.

For instance, the present invention also relates to a method for preventing and/or treating ulcer, digital ulcer, diabetic gangrene, diabetic foot ulcer, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance, connective tissue disease, chronic kidney diseases including glomerulonephritis and diabetic nephropathy at any stage, diseases in which fibrosis of organs or tissues is involved, or respiratory diseases, comprising administering the pharmaceutical formulation or dosage form as described above to a human patient in need thereof.

### EXAMPLES

### Abbreviations

API active pharmaceutical ingredient
EC ethyl cellulose
HPMC hydroxypropyl methylcellulose or hypromellose
HPMC-AS hydroxypropyl methylcellulose acetate succinate
HPMCP hydroxypropyl methyl cellulose phthalate
IR immediate release
MCC microcrystalline cellulose
PVA polyvinylalcohol
SR slow or sustained release

### Example 1: Process for the Production of Capsules Containing Slow Release Beads (SR Beads) with Active Pharmaceutical Ingredient Selexipag in Free Form

The manufacturing process for the capsules containing SR beads is conducted in the following steps:
1. Preparation of coating mixture 1 (API coating):
   a. Transfer methylene chloride and methanol into a suitable vessel.
   b. Add hypromellose 2910 5 mPa.s and selexipag to the solvent mixture, containing methylene chloride and methanol.
   c. Stir until both ingredients are dissolved.
2. Bead coating process 1 (layer 1)
   a. Transfer microcrystalline cellulose spheres into a suitable fluid bed coater.
   b. Spray the coating mixture (1) onto the microcrystalline cellulose spheres.
   c. After spraying, dry the obtained beads in the equipment.
   d. Collect the beads in a suitable container.
3. Preparation of coating mixture 2 (SR layer):
   a. Transfer methylene chloride, ethanol (96%) and dibutyl sebacate into a suitable vessel.
   b. Add ethylcellulose 20 mPa.s, and hypromellose 2910 5 mPa.s to the solvent mixture, containing methylene chloride and ethanol.
   c. Stir until a homogenous mixture is obtained.
4. Bead coating process 2 (layer 2):
   a. Transfer the beads obtained from step 2 into a suitable fluid bed coater.
   b. Spray the coating mixture (2) onto the beads.
   c. After spraying, dry the obtained beads in the equipment.
   d. Collect the beads in a suitable container.
5. Encapsulation of the SR beads:
   a. Fill the appropriate amount of SR coated beads in hard gelatin capsules size 3, red cap and body, using a suitable capsule filler.
   b. Close the capsules.
   c. Perform a 100% weight check on the filled capsules.
   d. Collect the filled capsules into a suitable bag.

Example 2: Sustained release beads and capsules, with a 75/25 ratio ethylcellulose/hypromellose polymer mixture, applied to a weight gain of 30% (w/w) of the total theoretical weight of the bulk beads. Oral capsules with 400 microgram selexipag.

As described above, the “weight gain” relates to the weight of the ethyl cellulose (EC) plus hydroxypropyl methylcellulose (HPMC) in the release rate controlling membrane coating based on the weight of the drug core.

TABLE 1

| | | | | |
|---|---|---|---|---|
| Component | Quality Reference | Funct ion | Quantity (mg) per g beads | Quantity (mg) per capsule |
| Core | | | | |
| MCC spheres | JPE | carrier | 677.78 | 51.85 |
| Coating mixture 1: first layer (API layer) | | | | |
| Selexipag | | API | 5.23 | 0 .40 |
| Hypromellose 2910 5 mPa.s | Ph. Eur. | Polymer | 52 .29 | 4.00 |
| Methylene chloride | Ph. Eur. | ^{a} Solvent | 496.73 | 38.00 |
| Methanol | Ph. Eur. | Solvent ^{a} | 496.73 | 38.00 |
| Coating mixture 2: second layer (release rate controlling coat) | | | | |
| Ethylcellulose 20 mPa.s | Ph. Eur. | Polymer for sustained release | 165.44 | 12.66 |
| Hypromellose 2910 5 mPa.s | Ph. Eur. | Polymer for sustained release | 55.15 | 4.22 |
| D ibutyl sebacate | USP/NF | Plasticizer | 44.12 | 3.38 |
| Methylene chloride | Ph. Eur. | Solvent ^{a} | 2047.06 | 156.60 |
| Ethanol (96%) | Ph. Eur. | Solvent ^{a} | 1364 .71 | 104 .40 |
| Total | | | 1000.00 mg | 76.50 mg ^{b} |
| Hard gelatin capsule ^{c} | | | | 1 capsule |

^{a} Process aid is removed during processing, does not appear in the final drug product
^{b} Theoretical amount: an assay correction may be applied
^{c} gelatin, iron oxide red, titanium dioxide

Example 3: Sustained release beads and capsules, with a 75/25 ratio ethylcellulose/hypromellose polymer mixture, applied to a weight gain of 20% (w/w) of the total theoretical weight of the bulk beads. Oral capsules with 400 microgram selexipag.

TABLE 2

| | | | | |
|---|---|---|---|---|
| Component | Quality Reference | Function | Quantity (mg) per g beads | Quantity (mg) per capsule |
| Core | | | | |
| MCC spheres | JPE | carrier | 729.53 | 117.60 |
| Coating mixture 1: first layer (API layer) | | | | |
| Selexipag | | API | 2.48 | 0.40 |
| Hypromellose 2910 5 mPa.s | Ph. Eur. | Polymer | 74.44 | 12.00 |
| Methylene chloride | Ph. Eur. | Solvent ^{a} | 1203.47 | 194.00 |
| Methanol | Ph. Eur. | Solvent ^{a} | 1203.47 | 194.00 |
| Coating mixture 2: second layer (release rate controlling coat) | | | | |
| Ethylcellulose 20 mPa.s | Ph. Eur. | Polymer for sustained release | 120.97 | 19.50 |
| Hypromellose 2910 5 mPa.s | Ph. Eur. | Polymer for sustained release | 40.32 | 6.50 |
| Dibutyl sebacate | USP/NF | Plasticizer | 32.26 | 5.20 |
| Methylene chloride | Ph. Eur. | Solvent ^{a} | 2303.23 | 371.28 |
| Ethanol (96%) | Ph. Eur. | Solvent ^{a} | 1535.48 | 247.52 |
| Total | | | 1000.00 mg | 161.20 mg ^{b} |
| Hard gelatin capsule ^{c} | | | | 1 capsule |

^{a} Process aid is removed during processing, does not appear in the final drug product
^{b} Theoretical amount: an assay correction may be applied
^{c} gelatin, iron oxide red, titanium dioxide

Example 4: Sustained release beads and capsules, with a 75/25 ratio ethylcellulose/hypromellose polymer mixture, applied to a weight gain of 10% (w/w) of the total theoretical weight of the bulk beads. Oral capsules with 400 microgram selexipag.

TABLE 3

| | | | | |
|---|---|---|---|---|
| Component | Qual ity Reference | Funct ion | Quantity (mg) per g beads | Quant ity (mg) per capsule |
| Core | | | | |
| MCC spheres | JPE | carr ier | 807.69 | 117.60 |
| Coating mixture 1: first layer (API layer) | | | | |
| Selexipag | | API | 2.75 | 0.40 |
| Hypromellose 2910 5 mPa.s | Ph. Eur. | Polymer | 82.42 | 12.00 |
| Methylene chloride | Ph. Eur. | Solvent ^{a} | 1332.42 | 194.00 |
| Methanol | Ph. Eur. | Solvent ^{a} | 1332.42 | 194.00: |
| Coating mixture 2:second layer (release rate controlling coat) | | | | |
| Ethylcellulose 20 mPa.s | Ph. Eur. | Polymer for sustained | 66.96 | 9.75 |
| | | release | | |
| Hypromellose 2910 5 mPa.s | Ph. Eur. | Polymer for sustained release | 22.32 | 3.25 |
| Dibutyl sebacate | USP/NF | Plasticizer | 17.86 | 2.60 |
| Methylene chloride | Ph. Eur. | Solvent ^{a} | 1275.00 | 185.64 |
| Ethanol (96%) | Ph. Eur. | Solvent^{a} | 850.00 | 123.76 |
| Total | | | 1000.00 mg | 145.60 mg ^{b} |
| Hard gelatin capsule ^{c} | | | | 1 capsule |

^{a} Process aid is removed during processing, does not appear in the final drug product
^{b} Theoretical amount: an assay correction may be applied
^{c} gelatin, iron oxide red, titanium dioxide

Example 5: Sustained release beads and capsules, with a 90/10 ratio ethylcellulose/hypromellose polymer mixture, applied to a weight gain of 10% (w/w) of the total theoretical weight of the bulk beads. Oral capsules with 400 microgram selexipag metabolite.

TABLE 4

| | | | |
|---|---|---|---|
| Component | Function | Quantity (mg) per g beads | Quantity (mg) per capsule |
| Core | | | |
| MCC | carrier | 807.661 | 117.482 |
| Coating mixture 1: first layer (API layer) | | | |
| Selexipag Metabolite | API | 2.747 | 0.400 |
| HPMC 2910 5 mPa.s | Polymer | 82.414 | 11.988 |
| methylene chloride, extra : pure | Solvent ^{a} | 1332 .366 | 193.806 |
| methanol, extra pure | Solvent ^{a} | 1332.366 | 193.806 |
| Coating mixture 2: second layer (release rate controlling coat) | | | |
| EC 20 mPa.s | Polymer for sustained release | 80.366 | 11.690 |
| HPMC 5 mPa.s | Polymer for sustained release | 8 .937 | 1.300 |
| dibutylsebacate | Plasticizer | 17.874 | 2.600 |
| methylene chloride | Solvent ^{a} | 1274 .921 | 185.450 |
| ethanol, anhydrous | Solvent ^{a} | 849.993 | 123.640 |
| Total | | 1000.000 | 145.460 |
| Hard gelatin capsule | | | 1 capsule |

### Example 6

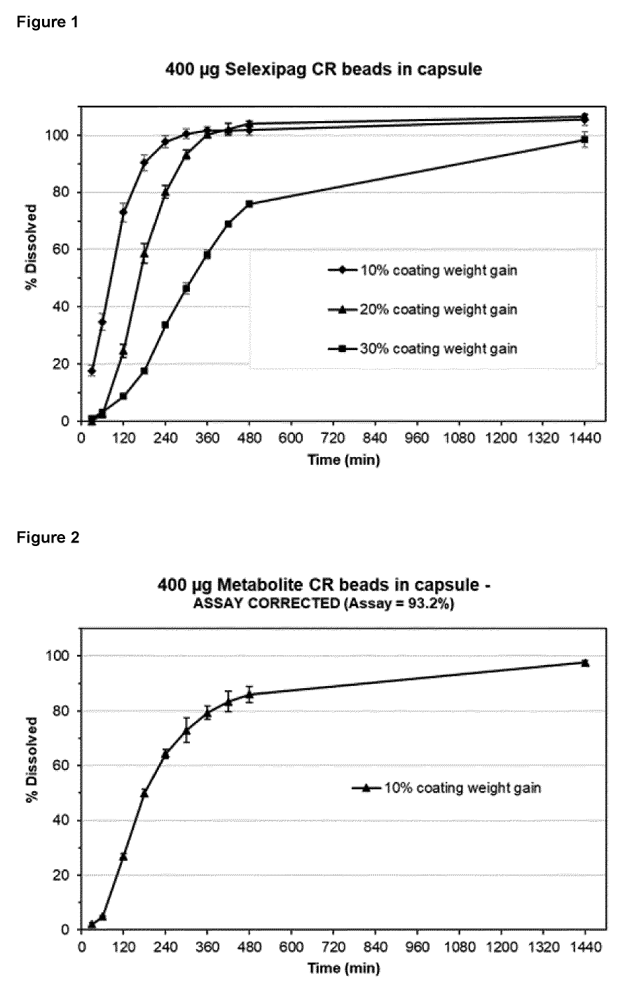

FIG. 1 shows a graph of an in vitro dissolution test of 400 microgram selexipag capsules containing the pharmaceutical formulation with 3 different release rate controlling membranes. Dissolution testing was performed in 0.05 M sodium phosphate buffer pH6.8 using the paddle apparatus at 37° C. The particles of the pharmaceutical formulation differed in the thickness of the release rate controlling membranes. In a first example, the weight of the ethyl cellulose (EC) plus hydroxypropyl methylcellulose (HPMC) in the release rate controlling membrane coating was 10 wt% (10% coating weight gain), in the second example, the weight of the ethyl cellulose (EC) plus hydroxypropyl methylcellulose (HPMC) in the release rate controlling membrane coating was 20 wt% (20% coating weight gain), and in the third example, the weight of the ethyl cellulose (EC) plus hydroxypropyl methylcellulose (HPMC) in the release rate controlling membrane coating was 30 wt% (30% coating weight gain), all based on the weight of the drug core.

FIG. 2 shows a graph of an in vitro dissolution test of a 400 microgram selexipag metabolite capsule containing the pharmaceutical formulation with a release rate controlling membrane. Dissolution testing was performed in 0.05 M sodium phosphate buffer pH6.8 using the paddle apparatus at 37° C. In the particles of the pharmaceutical formulation, the weight of the ethyl cellulose (EC) plus hydroxypropyl methylcellulose (HPMC) in the release rate controlling membrane coating was 10 wt% (10% coating weight gain), based on the weight of the drug core.

Example 6: A Study for Selexipag Sustained Release in Healthy Male Subjects is under investigation.

This study is a pilot formulation screening study conducted to identify and select an oral SR formulation providing PK profiles of selexipag and its metabolite ACT-333679 supporting a once daily dosing regimen.

To achieve this, the SR formulation should result in comparable daily exposure (area under the concentration-time curve [AUC]) as the equivalent daily dose of the IR formulation administered as two doses 12 hours apart, while the maximum plasma concentration (Cmax) for the SR formulation should be close to or below Cmax of the IR formulation and the plasma concentration 24 hours after dosing (C24h) of the SR formulation should be close to or higher than C24h for the IR formulation.

Pharmacokinetic parameters (PP) for selexipag and ACT 333679 combined (PPcombined), taking into account their different potencies, will be used for the primary comparison of SR and IR formulations.

Three different release profiles, designed as a fast (F), medium (M), and slow (S) release profile will be tested. Tested are sustained release pellets SRep as described in the present invention.

Development of a SR dosage form of selexipag supporting a once daily dosing regimen is expected to provide improved convenience for patients on selexipag treatment. As PAH is a chronic, progressive disease with significant morbidity and mortality, even small improvements in terms of clinical outcome may prove beneficial to both individual patients and the society by reducing the rate of hospitalization and disease progression.

The primary objective is to evaluate the PK of selexipag and ACT-333679 following single oral administration of the SRep of selexipag at a dose of 400 microgram, with 3 different release profiles, as compared to selexipag IR tablets in healthy male subjects.

The secondary objective is to evaluate the safety and tolerability of a single oral administration of the SRep selexipag formulations at a dose of 400 microgram, with 3 different release profiles, as compared to selexipag IR tablets in healthy male subjects.

There will be a total of 4 dosing visits for each subject; 3 dosing visits where the selexipag SR formulation with 3 different release profiles will be administered as well as one visit where selexipag IR is administered. The treatments are further specified in Table 4. Study drug will be administered on Day 1 of each treatment period. The treatment periods for each individual subject will be separated by a washout period of at least 7 days. The washout period starts after study drug administration in one treatment period and ends with study drug administration in the next treatment period.

TABLE 5

Treatment Overview

| No. of Subjects | Treatment (all doses will be taken orally on Day 1) | | |
|---|---|---|---|
| 12 | Treatment SRep-F: | Selexipag 400 µg SRep-F, single dose | Fasted |
| | Treatment SRep-M: | Selexipag 400 µg SRep-M, single dose | Fasted |
| | Treatment SRep-S: | Selexipag 400 µg SRep-S, single dose | Fasted |
| | Treatment IR | Selexipag 200 µg IR, two doses 12 hours apart; total daily dose of 400 µg | Fasted |

IR=immediate release; SRep= encapsulated sustained release pellets; F=fast release profile; M=medium release profile; S=slow release profile

Twelve healthy subjects will be randomly assigned to receive 4 single oral doses of selexipag (open-label) during 4 subsequent treatment periods. Predose on Day 1 of Treatment Period 1, subjects will be randomly allocated in a 1:1:1:1 ratio to one of 4 treatment sequences (see Table 5).

TABLE 6

Randomization Scheme

| | | | | |
|---|---|---|---|---|
| Sequence | Treatment Period 1 | Treatment Period 2 | Treatment Period 3 | Treatment Period 4 |
| 1 (n=3) | IR | SRep-F | SRep-M | SRep-S |
| 2 (n=3) | SRep-F | SRep-S | IR | SRep-M |
| 3 (n=3) | SRep-M | IR | SRep-S | SRep-F |
| 4 (n=3) | SRep-S | SRep-M | SRep-F | IR |

IR=immediate release; F=fast release profile; M=medium release profile; S=slow release profile

Study drug will be administered orally in the morning of Day 1 of each treatment period, between 8:00 and 11:00 AM under fasted conditions following an overnight fast of at least 10 hours, with 240 mL of noncarbonated water.

For the treatment with the IR formulation only, two oral doses of selexipag IR 200 µg will be given 12 hours apart. The first dose will be given in the morning of Day 1, as specified above, and the second dose will be given 12 hours later. The evening dose of selexipag IR will be administered with 240 mL of water.

Blood samples for determination of selexipag and ACT 333679 plasma concentrations will be collected at the time points indicated in the Time and Events Schedule.

A total of 17 PK samples will be collected after each individual dose of the SR formulations, whereas a total of 26 PK samples are needed to fully cover the two consecutive doses (administered 12 hours apart) of the IR formulation.

1. A pharmaceutical formulation comprising
particles comprising as active pharmaceutical ingredient 2-{4-[N-(5,6-diphenylpyrazin-2-yl)-N-isopropylamino]butyloxy}-N-(methylsulfonyl)acetamide (selexipag), or a pharmaceutically acceptable salt thereof, and/or 2-(4-((5,6-diphenylpyrazin-2-yl)(isopropyl)amino)butoxy)acetic acid (selexipag metabolite), or a pharmaceutically acceptable salt thereof, and a water soluble polymer, to form a drug core;
said particles being coated with a release rate controlling membrane coating comprising ethyl cellulose (EC) and hydroxypropyl methylcellulose (HPMC); and
optionally one or more protective coats.

2. The pharmaceutical formulation according to claim 1, wherein the weight-by-weight ratio of ethyl cellulose (EC) to hydroxypropyl methylcellulose (HPMC) in the release rate controlling membrane coating is 95 : 5 (w/w) to 50 : 50 (w/w).

3. The pharmaceutical formulation according to claim 1, wherein the release rate controlling membrane coating further comprises a plasticizer.

4. The pharmaceutical formulation according to claim 3, wherein the plasticizer is selected from the group consisting of dibutyl sebacate, diethyl phthalate, triethyl citrate and triacetin.

5. The pharmaceutical formulation according to claim 1, wherein the hydroxypropyl methylcellulose is HPMC 2910 5 mPa.s.

6. The pharmaceutical formulation according to claim 1, wherein the ethyl cellulose (EC) is ethyl cellulose (EC) 20.

7. The pharmaceutical formulation according to claim 1, wherein the weight of the ethyl cellulose (EC) plus hydroxypropyl methylcellulose (HPMC) in the release rate controlling membrane coating ranges from 5 wt% to 50 wt% based on the weight of the drug core.

8. The pharmaceutical formulation according to claim 1, wherein the water-soluble polymer is selected from the group consisting of
hydroxyalkyl alkylcellulose;
hydroxypropyl methylcellulose acetate succinate (HPMC-AS);
hydroxypropyl methyl cellulose phthalate (HPMCP) alkylcellulose;
hydroxyalkylcellulos;
carboxyalkylcellulose;
alkali metal salt of a carboxyalkylcellulose;
carboxyalkylalkylcellulose;
carboxyalkylcellulose ester;
starch;
pectin;
chitine derivative;
polysaccharide or alkali metal or ammonium salt thereof;
polyacrylic acid or a salt thereof;
polymethacrylic acid acids and or a salt thereof, or methacrylate copolymer;
polyvinylalcohol;
polyvinylpyrrolidone or a copolymer of polyvinylpyrrolidone with vinyl acetate; and
polyalkylene oxid.

9. The pharmaceutical formulation according to claim 1, wherein the water-soluble polymer is selected from the group consisting of
hydroxypropyl methylcellulose (HPMC); and
hydroxypropyl methylcellulose acetate succinate (HPMC-AS).

10. The pharmaceutical formulation according to claim 1, wherein the water-soluble polymer is hydroxypropyl methylcellulose HPMC 2910 5 mPa.s.

11. The pharmaceutical formulation according to claim 1, wherein the weight-by-weight ratio of the active pharmaceutical ingredient in free form to the water-soluble polymer is in the range of 1 : 0.5 to 1 : 100.

12. The pharmaceutical formulation according to claim 1, wherein the active pharmaceutical ingredient and the water-soluble polymer are layered or coated on an inert sphere.

13. The pharmaceutical formulation according to claim 12, wherein the inert spheres are spheres having a diameter of 250-1180 micrometer.

14. The pharmaceutical formulation according to claim 1, wherein the protective coat is a light protective coat and/or a seal coat.

15. The pharmaceutical formulation according to claim 1, wherein the protective coat lies between the drug core and the release rate controlling membrane coating, and/or wherein the protective coat lies on the release rate controlling membrane coating.

16. A dosage form comprising a therapeutically effective amount of the pharmaceutical formulation of claim 1.

17. The dosage form according to claim 16, wherein the dosage form is for oral administration once daily.

18. A pharmaceutical package suitable for commercial sale comprising a container, a dosage form as claimed in claim 16 and associated with said package written matter specifying how said dosage form should be administered.

19. A process of preparing a pharmaceutical formulation according to claim 1, comprising
(a) admixing the active pharmaceutical ingredient 2-{4-[N-(5,6-diphenylpyrazin- 2-yl)-N-isopropylamino]butyloxy}-N-(methylsulfonyl)acetamide (selexipag) or a pharmaceutically acceptable salt thereof, and/or 2-(4-((5,6-diphenylpyrazin-2-yl)(isopropyl)amino)butoxy)acetic acid (selexipag metabolite), or a pharmaceutically acceptable salt thereof, with a water-soluble polymer in an organic solvent to form a drug core;
(b) optionally applying a protective coat to the drug core;
(c) admixing ethyl cellulose (EC) and hydroxypropyl methylcellulose (HPMC), and optionally a plasticizer, in an organic solvent, and applying the release rate controlling membrane coating;
(d) optionally applying a protective coat to the particle comprising the release rate controlling membrane coating.

20. A pharmaceutical formulation obtainable by the process of claim 19.

21. (canceled)

22. The method of claim 23 for preventing or treating pulmonary arterial hypertension (PAH).

23. A method for preventing and/or treating ulcer, digital ulcer, diabetic gangrene, diabetic foot ulcer, pulmonary hypertension, pulmonary arterial hypertension, Fontan disease and pulmonary hypertension associated with Fontan disease, sarcoidosis and pulmonary hypertension associated with sarcoidosis, peripheral circulatory disturbance, connective tissue disease, chronic kidney diseases including glomerulonephritis and diabetic nephropathy at any stage, diseases in which fibrosis of organs or tissues is involved, or respiratory diseases, comprising administering the pharmaceutical composition according to claim 1 to a human subject in need thereof.

24. The pharmaceutical composition according to claim 8, wherein:
the hydroxyalkyl alkylcellulose is hydroxyethyl methylcellulose or hydroxypropyl methylcellulose (HPMC);
the hydroxypropyl methyl cellulose phthalate (HPMCP) alkylcellulose is methylcellulose;
the hydroxyalkylcellulose is hydroxymethylcellulose, hydroxyethylcellulose, hydroxypropylcellulose or hydroxybutylcellulose;
the carboxyalkylcellulose is carboxymethylcellulose;
the alkali metal salt of the carboxyalkylcellulose is sodium carboxymethylcellulose;
the carboxyalkylalkylcellulose is carboxymethylethylcellulose;
the pectine is carboxymethylamylopectine;
the chitine derivative is chitosan;
the polysaccharide is alginic acid or an alkali metal or ammonium salt thereof, carrageenan, galactomannan, traganth, agar agar, gummi arabicum, guar gummi or xanthan gummi; and
the polyalkylene oxide is polyethylene oxide, polypropylene oxide or a copolymer of ethylene oxide and propylene oxide.
